(19) United States
(12) Patent Application Publication
(10) Pub. No.: US 2025/0314561 A1
(43) Pub. Date: Oct. 9, 2025

(54) FULL-AUTOMATIC MOUSE BLOOD SAMPLING APPARATUS

(71) Applicant: Jilin University, Changchun City (CN)

(72) Inventors: Fushi QUAN, Changchun City (CN); Zhicheng LIN, Changchun City (CN); Bao YUAN, Changchun City (CN); Jian CHEN, Changchun City (CN)

(21) Appl. No.: 19/022,062

(22) Filed: Jan. 15, 2025

(30) Foreign Application Priority Data
Apr. 3, 2024 (CN) 202410401902.X

(51) Int. Cl. G01N 1/10; G01N 1/28

(52) U.S. Cl. CPC G01N 1/10; G01N 1/286; G01N 2001/2873

(57) ABSTRACT

A full-automatic mouse blood sampling apparatus includes: a carrier frame; a mouse holding tube disposed on the top of the carrier frame and having a first notch formed on the top of the mouse holding tube; a first carrier plate disposed inside the rear end of the mouse holding tube, a blood sampling port is provided in a top surface of the first carrier plate, a rotating plate is disposed inside the blood sampling port, a front end of the rotating plate is rotationally connected to a side wall of the blood sampling port, a tail restraining assembly is disposed on a top surface of the rotating plate, and a first drive assembly is disposed below the first carrier plate; a tail cutting assembly disposed on the rear side of the mouse holding tube; and a centrifuge tube disposed below a rear end of the rotating plate.

### CROSS REFERENCE TO RELATED APPLICATION

This application claims the priority of Chinese Patent Application No. 202410401902.X, filed with the Chinese Patent National Intellectual Property Administration on Apr. 3, 2024, which is incorporated herein by reference in its entirety as part of the present application.

### TECHNICAL FIELD

The present disclosure relates to the technical field of animal blood sampling apparatuses, in particular to a full-automatic mouse blood sampling apparatus.

### BACKGROUND

Mice are mammals belonging to the Mus genus in the rodent family. Tail clipping is a common method of blood sampling in mice. Mice have relatively long tails with rich blood vessels and lymphatic vessels. Tail clipping is less damage to mice and has less impact on their health and survival. At present, a blood collector needs to personally operate to complete the tail clipping for sampling blood. After a mouse is restrained, the blood collector controls the tail of the mouse by one hand, and clips off 1-2 mm of the tail of the mouse with scissors by the other hand, then puts the scissors down, takes out a centrifuge tube, and collects blood flowing out of the tail of the mouse into the centrifuge tube.

During the whole blood sampling process, the blood collector needs to control the tail of the mouse all the time by one hand, while changing to pick up different instruments by the other hand, which not only consumes the physical strength of the blood collector, but also causes contamination because the tail hair of the mouse is stained with blood as the tail of the mouse is out of restraint accidentally.

### SUMMARY

An objective of the present disclosure is to provide a full-automatic mouse blood sampling apparatus, which is able to liberate the hands of a blood collector and realize full-automatic mouse blood sampling. A tail restraining assembly can restrain the tail of a mouse such that the tail hair of the mouse will not be stained with blood, thus avoiding contamination.

In order to achieve the object described above, the present disclosure provides the following technical solutions. A full-automatic mouse blood sampling apparatus includes: a carrier frame; a mouse holding tube disposed on a top of the carrier frame, with a first notch formed at a top of a rear end of the mouse holding tube; a first carrier plate disposed inside the rear end of the mouse holding tube, wherein a blood sampling port is provided in a top surface of the first carrier plate, a rotating plate is disposed inside the blood sampling port, a front end of the rotating plate is rotationally connected to a side wall of the blood sampling port, a tail restraining assembly is disposed on a top surface of the rotating plate, and a first drive assembly configured to drive the rotating plate to rotate is disposed below the first carrier plate; a tail cutting assembly disposed on a rear side of the mouse holding tube; and a centrifuge tube disposed below a rear end of the rotating plate.

In some embodiments, the tail cutting assembly includes: a first connecting plate with a first end located below the mouse holding tube and a second end located above the mouse holding tube; a first electric telescopic rod fixedly disposed on the carrier frame, an output end of the first electric telescopic rod being fixedly connected to the first end of the first connecting plate; a second connecting plate disposed on the second end of the first connecting plate and provided with a cutting blade and a guard plate; a severed-tail cleaning assembly disposed on the second end of the first connecting plate, a collection box being disposed on the first carrier plate and having an opening top surface; a compression hemostasis assembly disposed on the second end of the first connecting plate; and a blocking plate disposed on the second end of the first connecting plate.

In some embodiments, the second end of the first connecting plate is provided with a protruding plate, a first end of the protruding plate is fixedly connected to the first connecting plate, and a second end of the protruding plate extends above the first carrier plate; the severed-tail cleaning assembly includes a push rod and a stop lever, a first end of the push rod is fixedly connected to the protruding plate, a second end of the push rod is fixedly provided with a push block, the push block has a T-shaped cross-section, the stop lever is disposed on a rear side of the push rod, a gap is formed between the stop lever and the push rod, the stop lever has a length greater than that of the push rod, a first end of the stop lever is fixedly connected to the protruding plate, and a second end of the stop lever is located on a left side of the push block; and the collection box is located in front of the stop lever and is removably connected to the first connecting plate.

In some embodiments, the second connecting plate is located above the severed-tail cleaning assembly, a first end of the second connecting plate is fixedly connected to a middle of the push rod and stop lever, the cutting blade and the guard plate are located on a second end of the second connecting plate, the guard plate is located between the cutting blade and the tail restraining assembly, the guard plate has an L-shaped cross-section, and the guard plate and the cutting blade are removably connected to the second connecting plate.

In some embodiments, the blocking plate is disposed below the protruding plate and is fixedly connected to a bottom of the protruding plate, a shape of the blocking plate matches with a shape of a cross-section of the rear end of the mouse holding tube, a length of the blocking plate is greater than a width of the rear end of the mouse holding tube, and a front surface of the blocking plate is capable of sliding along a rear end surface of the mouse holding tube.

In some embodiments, the compression hemostasis assembly includes a press plate disposed in front of the push rod, a gap is formed between the press plate and the push rod, the press plate is fixedly connected to the protruding plate, gauze is disposed on a front side wall of the press plate, and a gap is formed between the press plate and the guard plate.

In some embodiments, the tail restraining assembly includes: a resilient metal sheet disposed on one side of the top surface of the rotating plate, a first end of the resilient metal sheet being rotationally connected to the rotating plate, a pull plate being disposed on a top surface of the resilient metal sheet, and a top surface of the pull plate being higher than a top surface of the press plate; and a limiting block fixedly disposed on an other side of the top surface of the rotating plate, a front side wall of the limiting block being provided with a clamping slot, a width of the clamping slot being gradually decreased from front to back, a width of a front end of the clamping slot being greater than a thickness of the resilient metal sheet; and the tail cutting assembly further includes a tail releasing assembly including: a second electric telescoping rod fixedly disposed on a top of the first end of the second connecting plate; and a push plate disposed in front of the press plate and fixedly connected to an output end of the second electric telescopic rod, a bottom surface of the push plate being lower than the top surface of the press plate, and a width of the push plate being greater than a width of the pull plate.

In some embodiments, the carrier frame includes a confining plate and a support plate, the confining plate has an opening front end surface, a second notch is formed at a top of a rear end of the confining plate, a front end of the mouse holding tube is located within the confining plate and fixedly connected to the confining plate, and the rear end of the mouse holding tube extends out of the confining plate through the second notch; and the support plate is disposed below the rear end of the mouse holding tube, a top of the support plate is fixedly connected to the mouse holding tube, and a bottom of the support plate is fixedly connected to the confining plate.

In some embodiments, the mouse holding tube is provided with a curved cover plate, the curved cover plate is disposed in the first notch and located in a middle of the mouse holding tube, and the curved cover plate is hinged to the mouse holding tube; and a third notch is formed in a bottom surface of the front end of the mouse holding tube, a second carrier plate is disposed within the third notch, and the carrier frame is provided with a second drive assembly configured to drive the second carrier plate to move up and down.

In some embodiments, the full-automatic mouse blood sampling apparatus further includes: a body adapting assembly disposed at the front end of the mouse holding tube; a body restraining assembly disposed on the curved cover plate; and a tail squeezing assembly disposed on the top of the mouse holding tube and located on the rear side of the curved cover plate.

Compared with the conventional technology, the present disclosure has the following beneficial effects:
(1) According to the present embodiments, the mouse blood sampling can be completed full-automatically, and the blood collector does not need to restrain the mouse by hands such that the hands of the blood collector can be liberated. The tail restraining assembly can restrain the rear end of the tail to prevent the tail moving around such that the tail hair of the mouse will not be stained with blood, thus avoiding contamination.
(2) In the present embodiments, the tail cutting assembly includes the severed-tail cleaning assembly, the compression hemostasis assembly and the tail releasing assembly, and the severed-tail cleaning assembly can clean up the cut-off tail end, avoiding that the cut-off tail end stays on the first carrier plate causing an obstruction to the compression hemostasis assembly. The compression hemostasis assembly can perform compression hemostasis on a cut of the tail after the blood sampling is completed. The tail releasing assembly may release the tail, and then the mouse can move.
(3) The present embodiment further includes the body adapting assembly, the body retraining assembly and the tail squeezing assembly. The body adapting assembly is located at the front end of the mouse holding tube, and can adjust the size of an internal space of the mouse holding tube to adapt to the mice having different body sizes. The mouse holding tube is provided with the curved cover plate hinged to the mouse holding tube. Since the body retraining assembly is disposed on the curved cover plate, the body of the mouse is fixed under the curved cover plate and cannot move, preventing the mouse from moving during the tail clipping for sampling blood. The tail squeezing assembly is disposed on the top of the mouse holding tube and located on the rear side of the curved cover plate. The tail squeezing assembly can press on the tail such that the blood flows out quickly from the cut of the tail.

### BRIEF DESCRIPTION OF THE DRAWINGS

FIG. 1 is an isometric diagram of a full-automatic mouse blood sampling apparatus according to the present disclosure when no mouse is placed thereon;

FIG. 2 is an isometric diagram of the full-automatic mouse blood sampling apparatus according to the present disclosure when a mouse is placed thereon;

FIG. 3 is an isometric diagram of the full-automatic mouse blood sampling apparatus according to the present disclosure after the body and tail of a mouse is restrained;

FIG. 4 is an isometric diagram of the full-automatic mouse blood sampling apparatus according to the present disclosure when a mouse is subjected to tail clipping for sampling blood;

FIG. 5 is an enlarged view of part A in FIG. 4;

FIG. 6 is a front sectional view of the full-automatic mouse blood sampling apparatus according to the present disclosure when a mouse is subjected to tail clipping for sampling blood;

FIG. 7 is an enlarged view of part B in FIG. 6;

FIG. 8 is an isometric diagram of the full-automatic mouse blood sampling apparatus according to the present disclosure when compression hemostasis is performed on a mouse;

FIG. 9 is an enlarged view of part C in FIG. 8.

FIG. 10 is an isometric diagram of the full-automatic mouse blood sampling apparatus according to the present disclosure when a mouse is released therefrom;

FIG. 11 is an isometric diagram of a carrier frame according to the present disclosure, viewed from one angle;

FIG. 12 is an isometric diagram of a carrier frame according to the present disclosure, viewed from another angle;

FIG. 13 is an isometric diagram of a mouse holding tube according to the present disclosure;

FIG. 14 is an isometric diagram of a body adapting assembly according to the present disclosure;

FIG. 15 is a front sectional view of a tail squeezing assembly according to the present disclosure;

FIG. 16 is an isometric diagram of a first carrier plate before rotation of a rotating plate according to the present disclosure;

FIG. 17 is an isometric diagram of the first carrier plate after rotation of the rotating plate according to the present disclosure;

FIG. 18 is a front view of a limiting block according to the present disclosure;

FIG. 19 is an isometric diagram of a tail cutting assembly according to the present disclosure;

FIG. 20 is an isometric diagram of a first connecting plate according to the present disclosure;

FIG. 21 is an isometric diagram of a blocking plate according to the present disclosure;

FIG. 22 is an isometric diagram of a severed-tail cleaning assembly and a compression hemostasis assembly according to the present disclosure;

FIG. 23 is an isometric diagram of a second connecting plate according to the present disclosure;

FIG. 24 is a front view of the second connecting plate according to the present disclosure.

### REFERENCE SIGNS INCLUDES

1—carrier frame, 11—confining plate, 111—second notch, 112—through slot, 12—support plate, 13—second drive assembly, 131—third electric telescopic rod, 132—drive plate, 2—mouse holding tube, 21—first notch, 22—curved cover plate, 23—third notch, 24—second carrier plate, 25—body adapting assembly, 251—mouse blocking plate, 2511—air hole, 252—fourth electric telescopic rod, 253—third connecting plate, 26—body restraining assembly, 261—air pump, 262—air bag, 263—air pipe, 27—tail squeezing assembly, 271—vertical plate, 272—second motor, 273—rotating box, 2731—through hole, 274—sliding plate, 275—spring, 276—press rod, 277—pinch roller, 2771—curved groove, 28—tube holding slot, 29—clamping hoop, 210—laser sensor, 3—first carrier plate, 31—blood sampling port, 32—rotating plate, 321—insert, 33—tail restraining assembly, 331—resilient metal sheet, 332—pull plate, 333—limiting block, 3331—clamping slot, 34—first drive assembly, 341—first gear, 342—first motor, 343—second gear, 35—collection box, 36—slot, 4—tail cutting assembly, 41—first connecting plate, 411—protruding plate, 42—first electric telescopic rod, 43—second connecting plate, 44—cutting blade, 45—guard plate, 46—severed-tail cleaning assembly, 461—push rod, 4611—push block, 462—stop lever, 47—compression hemostasis assembly, 471—press plate, 472—gauze, 48—blocking plate, 49—tail releasing assembly, 491—second electric telescopic rod, 492—push plate, 5—centrifuge tube, 6—mouse, 61—tail, and 7—base plate.

### DETAILED DESCRIPTION OF THE EMBODIMENTS

The technical solutions in the embodiments of the present disclosure will be clearly and completely described below in conjunction with the accompanying drawings in the embodiments of the present disclosure. Apparently, the embodiments described are merely some rather than all of the embodiments of the present disclosure. On the basis of the embodiments in the present disclosure, all the other embodiments that would have been obtained by those of ordinary skill in the art without involving any inventive effort shall fall within the scope of protection of the present disclosure.

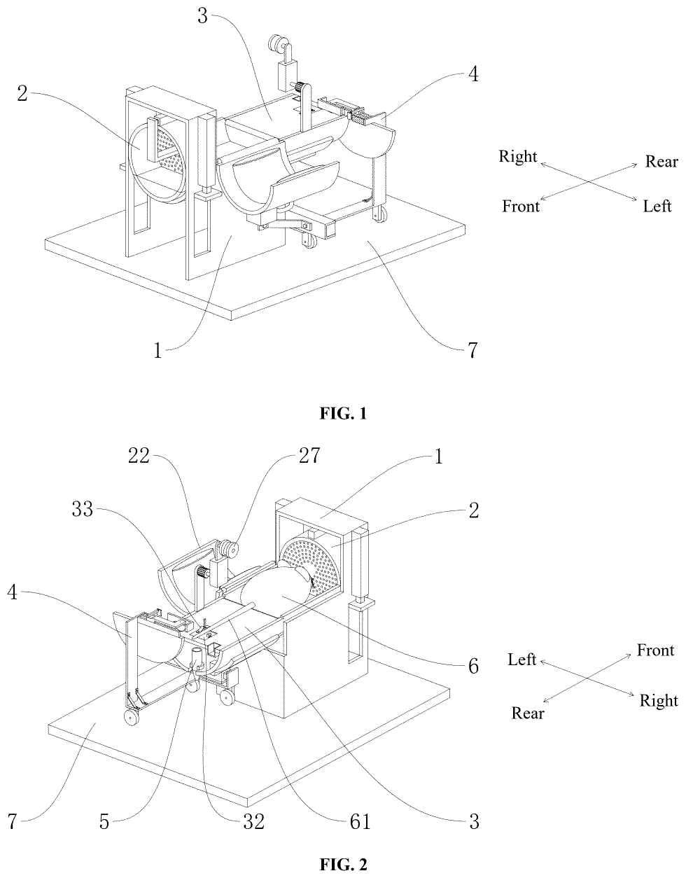

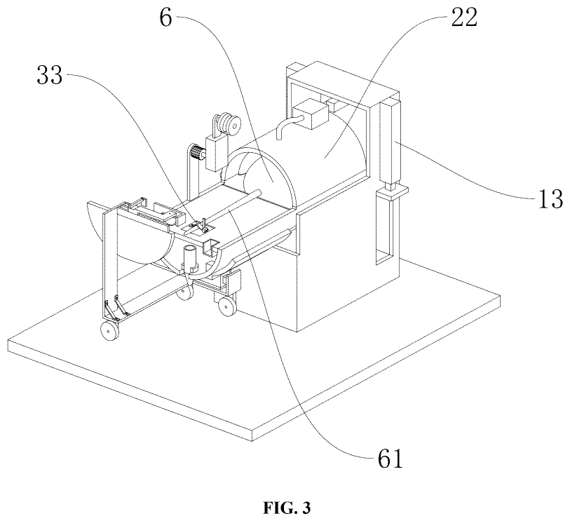

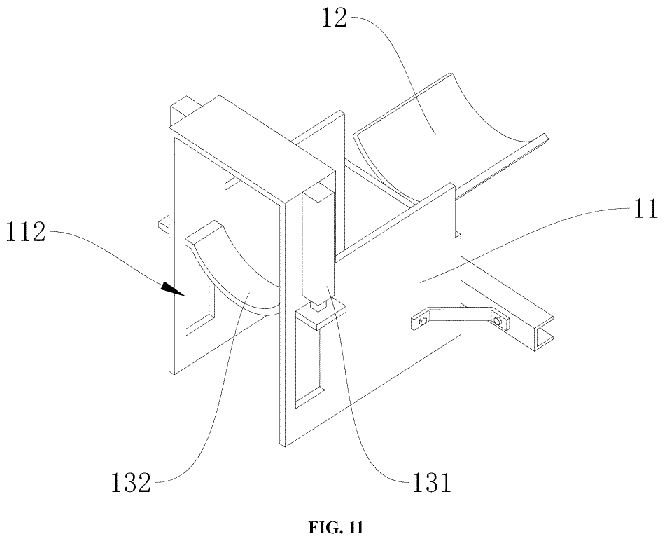

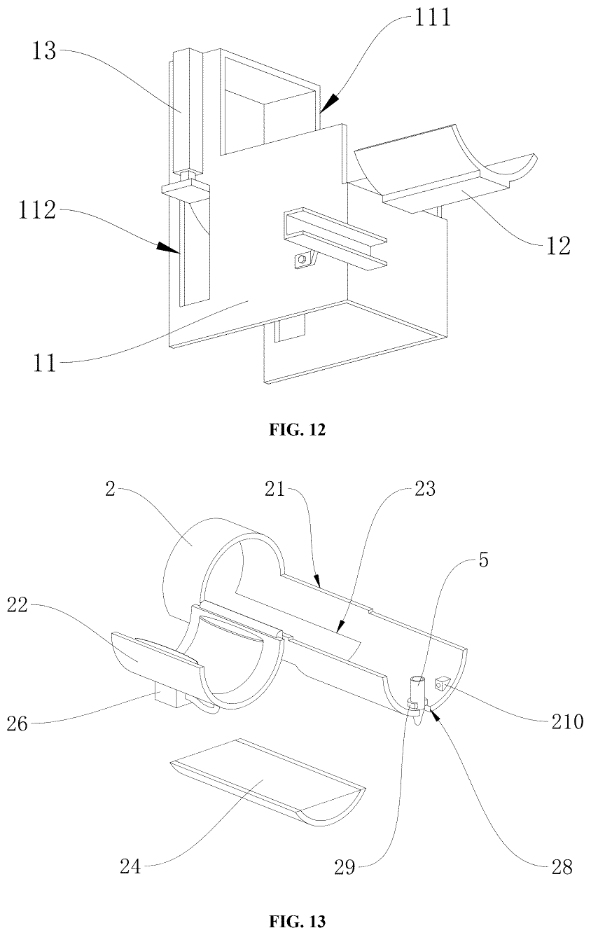

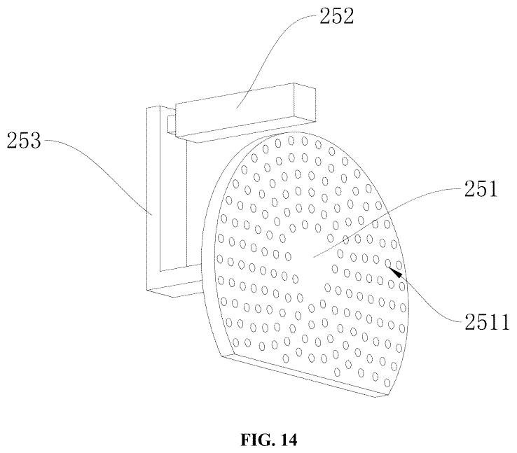

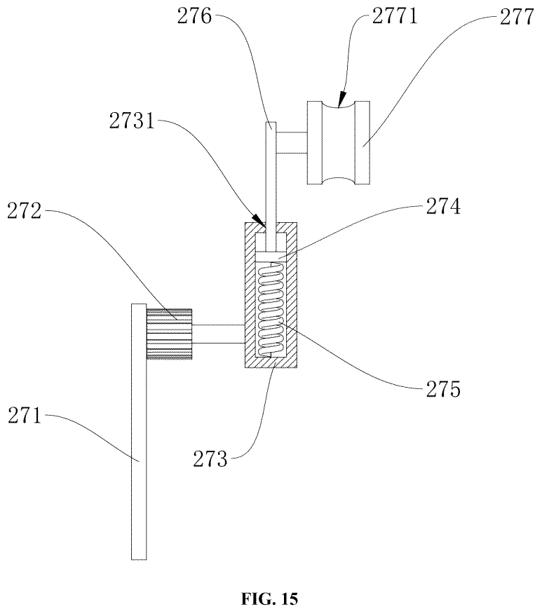

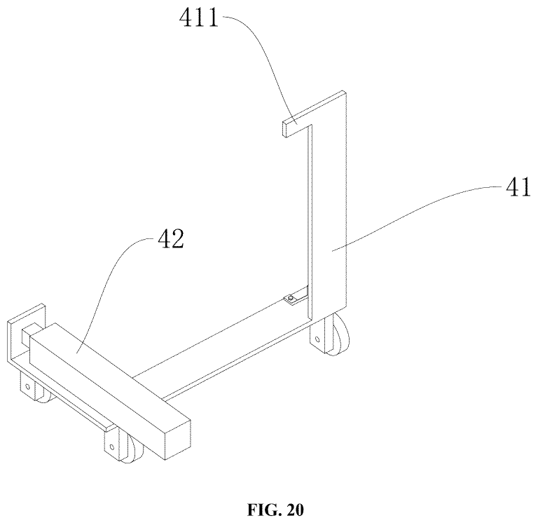

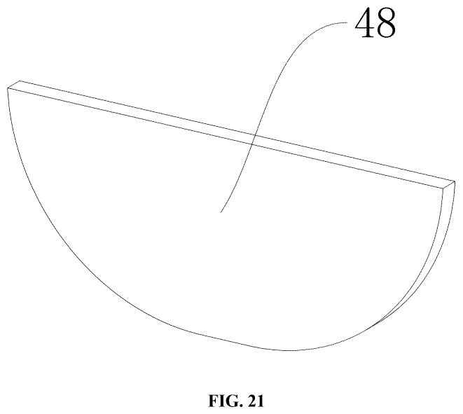

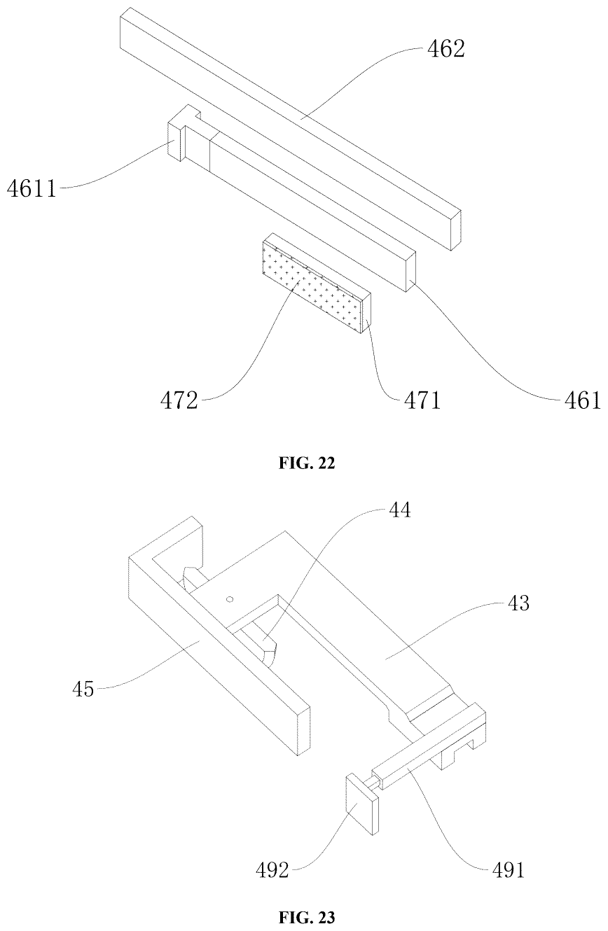

Referring to FIGS. 1 to 24, the present disclosure provides a technical solution as below. A full-automatic mouse blood sampling apparatus includes a carrier frame 1, a mouse holding tube 2, a first carrier plate 3, a tail cutting assembly 4, and a centrifuge tube 5. The carrier frame 1 is fixedly disposed on the base plate 7, and the base plate 7 may be fixed to an operation table by bolting or welding. The mouse holding tube 2 is disposed on the top of the carrier frame 1, and a first notch 21 is formed at the top of a rear end of the mouse holding tube 2. Before performing tail clipping for sampling blood of a mouse 6, a blood collector first puts the mouse 6 into the mouse holding tube 2 through the first notch 21, with the head of the mouse 6 facing forward and a tail 61 facing backward (the front-rear and left-right directions are as shown in FIGS. 1 to 2 and FIG. 19). The first carrier plate 3 is disposed inside the rear end of the mouse holding tube 2, where a blood sampling port 31 is provided in a top surface of the first carrier plate 3, a rotating plate 32 is disposed inside the blood sampling port 31, and a front end of the rotating plate 32 is rotationally connected to a side wall of the blood sampling port 31. The tail 61 of the mouse 6 is placed on the first carrier plate 3, and the end of the tail 61 extends to a rear side of the rotating plate 32. At this time, the rotating plate 32 is not rotated, and a top surface of the rotating plate 32 is flush with the top surface of the first carrier plate 3. A tail restraining assembly 33 is disposed on a top surface of the rotating plate 32, and the tail cutting assembly 4 is disposed on a rear side of the mouse holding tube 2. Referring to FIG. 3, the blood collector restrains a rear end of the tail 61 by the tail restraining assembly 33, and thus the tail 61 will not move around.

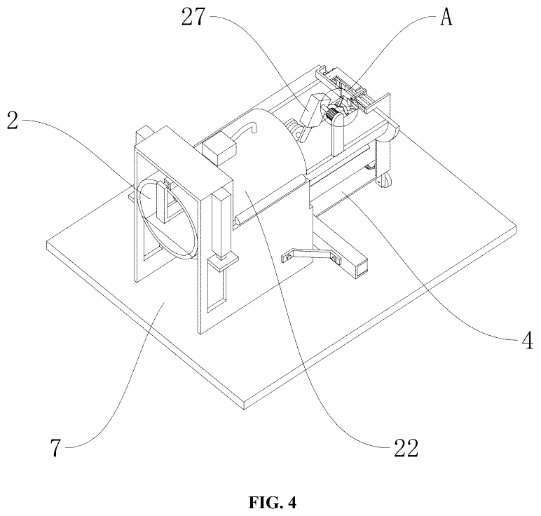

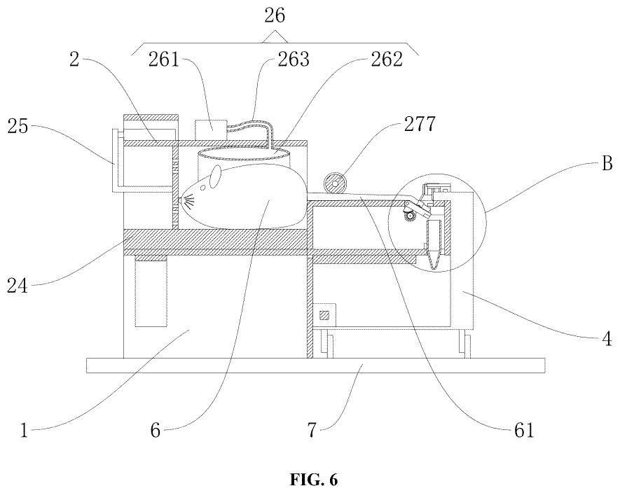

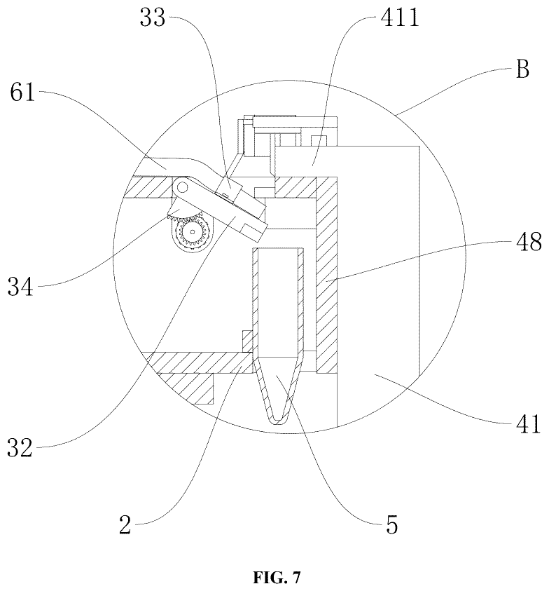

Then, the mouse is subjected to the tail clipping for sampling blood, in which the tail cutting assembly 4 works first to cut off the end of the tail 61. Referring to FIGS. 6 and 7, a first drive assembly 34 configured to drive the rotating plate 32 to rotate is disposed below the first carrier plate 3, then the first drive assembly 34 works to drive the rotating plate 32 to rotate downward, and the rotating plate 32 will drive the rear end of the tail 61 to rotate downward together. The centrifuge tube 5 is located below a rear end of the rotating plate 32, and the centrifuge tube 5 is configured for collecting blood of the mouse 6. Referring to FIGS. 4 to 7, after the rotating plate 32 is rotated downward, the rear end of the tail 61 is rotated downward, passes through the blood sampling port 31 and moves to the bottom of the first carrier plate 3, and at this time, a cut of the tail 61 just faces a top opening of the centrifuge tube 5, and the blood flowing out of the tail 61 drips downward to the centrifuge tube 5 for collection, thus completing the sampling blood from the mouse 6.

According to the present disclosure, the blood sampling of the mouse 6 can be completed full-automatically, and the blood collector does not need to restrain the mouse 6 by hands such that the hands of the blood collector can be liberated. The tail restraining assembly 33 may restrain the rear end of the tail 61 and prevent the tail 61 moving around such that the tail hair of the mouse 6 will not be stained with blood, thus avoiding contamination.

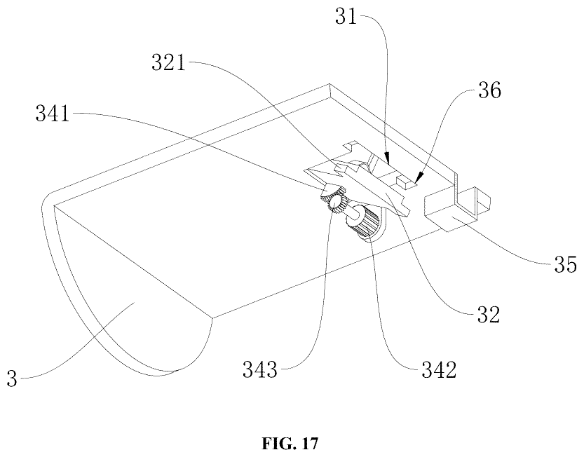

Referring to FIG. 17, an insert 321 is disposed at the bottom of each of the left and right sides of the rear end of the rotating plate 32, and a slot 36 is provided in a bottom surface of the first carrier plate 3 and located at each of the left and right sides of the blood sampling port 31, and the slot 36 corresponds to the insert 321 in position. After the insert 321 is inserted to the slot 36, the top surface of the rotating plate 32 is flush with the top surface of the first carrier plate 3, that is, the insert 321 fits with the slot 36 to restrain the rotating plate 32 from rotating to protrude from the top of the rotating plate 32. Referring to FIG. 17, the first drive assembly 34 includes a first gear 341, a first motor 342, and a second gear 343. The first gear 341 is fixedly disposed at a front end of a bottom surface of the rotating plate 32, and the first gear 341 is an incomplete gear; the first motor 342 is fixedly disposed on the bottom surface of the first carrier plate 3; and the second gear 343 is fixedly disposed at an output end of the first motor 342 and is meshed with the first gear 341.

Referring to FIGS. 4 to 7 and FIG. 17, the first drive assembly 34 works after the tail 61 is cut off, the first motor 342 drives the second gear 343 to rotate, and the second gear 343 drives the rotating plate 32 to rotate downward through the first gear 341 so as to drive the cut of the tail 61 to move toward the top opening of the centrifuge tube 5. Referring to FIG. 13, a tube holding slot 28 is formed in the bottom of the rear end of the mouse holding tube 2, a clamping hoop 29 is fixedly disposed at the top of the tube holding slot 28, and the centrifuge tube 5 is disposed in the clamping hoop 29 and is removably connected to the clamping hoop 29. A laser sensor 210 is disposed inside the rear end of the mouse holding tube 2, and the laser sensor 210 is capable of sensing the level of the blood inside the centrifuge tube 5. After the blood of the mouse 6 is collected by the centrifuge tube 5, the first motor 342 drives the second gear 343 to rotate reversely, which ultimately drives the rotating plate 32 to rotate upward, thereby driving the tail 61 to move over the first carrier plate 3. After that, the centrifuge tube 5 can be removed from the clamping hoop 29.

Referring to FIGS. 1, 2, 19, and 23, the tail cutting assembly 4 includes a first connecting plate 41, a first electric telescopic rod 42, a second connecting plate 43, and a cutting blade 44. When the rear end of the tail 61 is fixedly restrained by the tail restraining assembly 33, the cutting blade 44 is located on a left side of the tail 61 and the rear side of the rotating plate 32, and a bottom end of the cutting blade 44 abuts against the top surface of the first carrier plate 3. During operation of the tail cutting assembly 4, the first electric telescopic rod 42 extends to drive the first connecting plate 41 to move to the right side, and the first connecting plate 41 then drives the cutting blade 44 to move to the right side along the top surface of the first carrier plate 3 through the second connecting plate 43. In the process, the cutting blade 44 cuts off the end of the tail 61. Referring to FIGS. 2, 19 and 20, multiple rollers are arranged at the bottom of the first connecting plate 41. By means of the rollers, the first connecting plate 41 moves smoothly on a top surface of the base plate 7, and the friction is reduced.

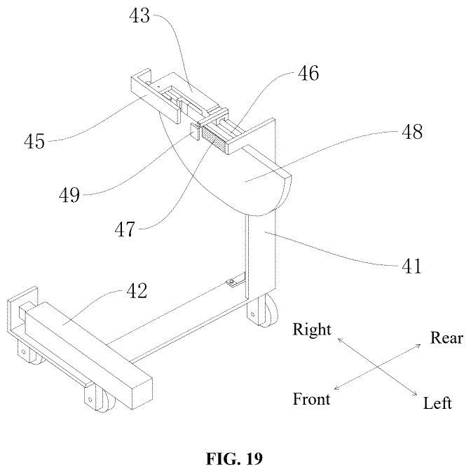

Referring to FIGS. 19 and 23, the tail cutting assembly 4 further includes a guard plate 45, a severed-tail cleaning assembly 46, a compression hemostasis assembly 47, and a blocking plate 48. When the blood collector restrains the tail 61 by means of the tail restraining assembly 33, the guard plate 45 can serve as a spacer to prevent the hand of the blood collector from being scratched by the cutting blade 44. The severed-tail cleaning assembly 46, the compression hemostasis assembly 47 and the blocking plate 48 are all disposed on the second end of the first connecting plate 41, and thus will move together with the first connecting plate 41. The severed-tail cleaning assembly 46 may clean up the cut-off tail end, avoiding that the cut-off tail end stays on the first carrier plate 3 causing an obstruction to the compression hemostasis assembly 47. The compression hemostasis assembly 47 may compress the cut of the tail 61 to stop bleeding after the blood sampling is completed. The blocking plate 48 may block the rear end of the mouse holding tube 2 to prevent external dust from falling into the centrifuge tube 5 through the opening at the rear end of the mouse holding tube 2 in the process of collecting blood from the mouse 6.

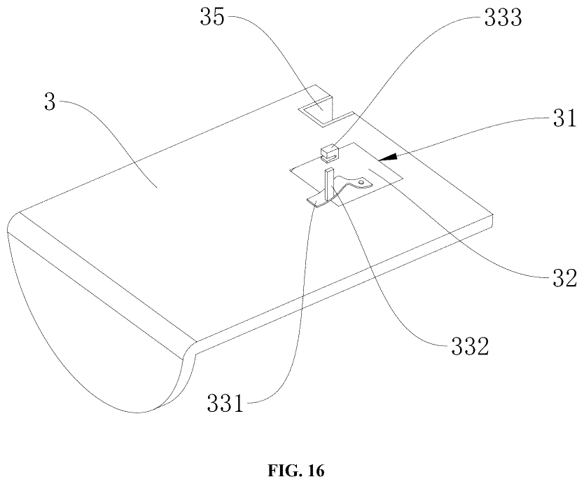

Referring to FIGS. 1 to 4 and FIG. 20, the second end of the first connecting plate 41 is provided with a protruding plate 411, a first end of the protruding plate 411 is fixedly connected to the first connecting plate 41, and a second end of the protruding plate 411 extends above the first carrier plate 3. Referring to FIG. 19 and FIGS. 22 and 23, the severed-tail cleaning assembly 46 includes a push rod 461 and a stop lever 462, and a first end of the push rod 461 is fixedly connected to the protruding plate 411. The second end of the push rod 461 is fixedly provided with a push block 4611. The push block 4611 has a T-shaped cross-section. The severed-tail and cleaning assembly 46 is disposed on a left rear side of the cutting blade 44, so that when the first electric telescopic rod 42 extends, the cutting blade 44 first cuts the tail 61, the first connecting plate 41 continues to move to the right side, and the severed-tail and cleaning assembly 46 then cleans up the end of the tail that is cut off. Referring to FIGS. 16 and 17, a collection box 35 is disposed on the first carrier plate 3, and the collection box 35 has an opening top surface. The collection box 35 is located in front of the stop lever 462. The collection box 35 is removably connected to the first connecting plate 41.

During operation of the severed-tail cleaning assembly 46, the first connecting plate 41 drives the stop lever 462 to move to the right side through the protruding plate 411 to block the rear edges of the mouse holding tube 2 and the collection box 35, and at the same time, the protruding plate 411 drives the push block 4611 through the push rod 461 to move to the right side so as to push the cut-off tail end into the collection box 35, avoiding that the cut-off tail end stays on the first carrier plate 3 causing an obstruction to the compression hemostasis assembly 47.

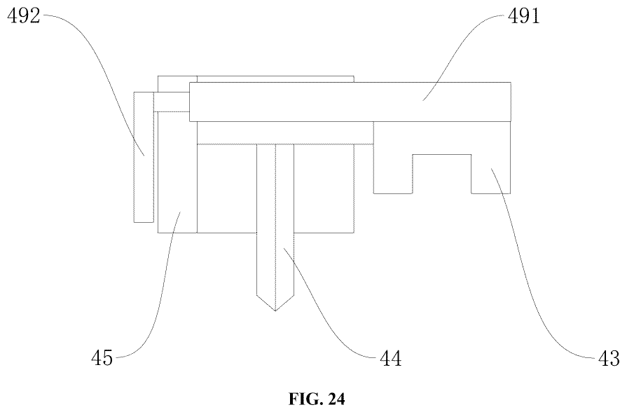

Referring to FIGS. 1 and 2, FIG. 19, and FIGS. 22 and 23, the second connecting plate 43 is located above the severed-tail cleaning assembly 46, and a first end of the second connecting plate 43 is fixedly connected to the middle of the push rod 461 and stop lever 462. The cutting blade 44 and the guard plate 45 are both disposed on the second end of the second connecting plate 43. The guard plate 45 is located between the cutting blade 44 and the tail restraining assembly 33, and the guard plate 45 has an L-shaped cross section, so that regardless of whether the hand of the blood collector is close to the cutting blade 44 from the front side or the right side, the guard plate 45 can shield and protect the hand of the blood collector. Both the guard plate 45 and the cutting blade 44 are removably connected to the second connecting plate 43, and the guard plate 45 and the cutting blade 44 may be removed for replacement. Referring to FIG. 24, a bottom surface of the guard plate 45 is higher than the bottom of the cutting blade 44, so that in the process of cutting the tail 61 by means of the cutting blade 44, the guard plate 45 will not come into contact with the tail 61, avoiding affecting the normal operation of the cutting blade 44.

Referring to FIGS. 19 to 21, the blocking plate 48 is disposed below the protruding plate 411 and is fixedly connected to the bottom of the protruding plate 411. Referring to FIGS. 1 to 3, FIG. 13 and FIG. 21, the shape of the blocking plate 48 matches with the shape of the cross-section of the rear end of the mouse holding tube 2, a length of the blocking plate 48 is greater than a width of the rear end of the mouse holding tube 2, and a front surface of the blocking plate 48 is capable of sliding along a rear end surface of the mouse holding tube 2. Referring to FIGS. 3 to 5, after the cutting blade 44 cuts off the tail 61, i.e., when the blood of the mouse 6 is collected by means of the centrifuge tube 5, the blocking plate 48 moves to the right side along with the protruding plate 411, the blocking plate 48 blocks the opening at the rear end of the mouse holding tube 2 fully, and the rightmost end of the blocking plate 48 is completely aligned with the rightmost end of the rear end of the mouse holding tube 2. However, because the length of the blocking plate 48 is greater than the width of the rear end of the mouse holding tube 2, the left end of the blocking plate 48 at this point still protrudes from the leftmost end of the rear end of the mouse holding tube 2.

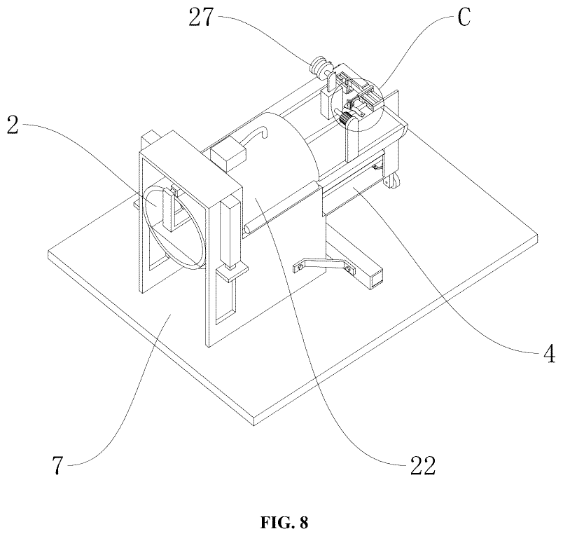

Referring to FIG. 8, when the compression hemostasis assembly 47 performs compression hemostasis on the cut of the tail 61, the blocking plate 48 has continued to move to the right side by a certain distance along with the protruding plate 411, and at this time, the leftmost end of the blocking plate 48 is completely aligned with the leftmost end of the rear end of the mouse holding tube 2, while the right end of the blocking plate 48 protrudes from the rightmost end of the rear end of the mouse holding tube 2. That is, when the compression hemostasis is performed on the cut of the tail 61, the opening at the rear end of the mouse holding tube 2 will not be exposed.

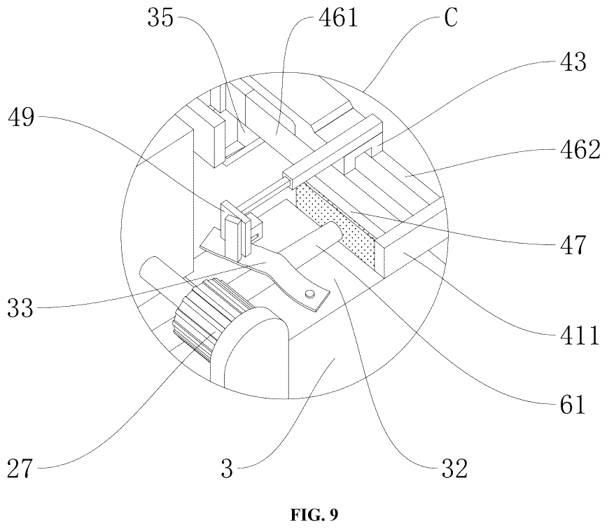

Referring to FIGS. 19 to 20 and FIG. 22, the compression hemostasis assembly 47 includes a press plate 471, the press plate 471 is disposed in front of the push rod 461, and a gap is provided between the press plate 471 and the push rod 461. The press plate 471 is fixedly connected to the protruding plate 411, gauze 472 is disposed on a front side wall of the press plate 471, and a gap is provided between the press plate 471 and the guard plate 45. Referring to FIGS. 8 and 9, when the blood of the mouse 6 is collected by means of the centrifuge tube 5, the first drive assembly 34 works to drive the rotating plate 32 to rotate upward, the top surface of the rotating plate 32 is flush with the top surface of the first carrier plate 3, the first electric telescopic rod 42 continues to extend to drive the first connecting plate 41 to continue to move to the right side, at which time the protruding plate 411 drives the press plate 471 to move to the rear side of the tail 61, the gauze 472 on a front surface of the press plate 471 abuts against the cut of the tail 61 to stop bleeding of the tail 61 by compression. The gauze 472 is removably connected to the press plate 471, and accordingly it can be replaced.

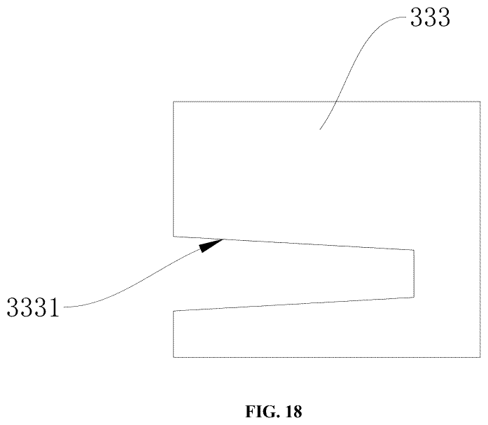

Referring to FIG. 16, the tail restraining assembly 33 includes a resilient metal sheet 331 and a limiting block 333. The resilient metal sheet 331 and the limiting block 333 are fixedly disposed on both sides of the top surface of the rotating plate 32, respectively. A first end of the resilient metal sheet 331 is rotationally connected to the rotating plate 32, and a pull plate 332 is disposed on a top surface of the resilient metal sheet 331. Referring to FIG. 18, a front side wall of the limiting block 333 is provided with a clamping slot 3331, a width of the clamping slot 3331 is gradually decreased from front to back, a width of a front end of the clamping slot 3331 is greater than a thickness of the resilient metal sheet. 331.

Referring to FIGS. 2 and 3, when the mouse 6 is placed in the mouse holding tube 2, the pull plate 332 is first pushed forward to rotate the resilient metal sheet 331 away from the clamping slot 3331, and then the tail 61 of the mouse 6 is placed on the rotating plate 32, with the end of the tail 61 extending to the rear side of the rotating plate 32. Then, the pull plate 332 is pushed reversely. When the resilient metal sheet 331 presses the tail 61 from the above, the resilient metal sheet 331 is then pushed into the clamping slot 3331, and the resilient metal sheet 331 is secured by the limiting block 333. At this time, the rear end of the tail 61 is restrained by the resilient metal sheet 331 to prevent the tail 61 moving around such that the tail hair of the mouse 6 will not be stained with blood, thus avoiding contamination.

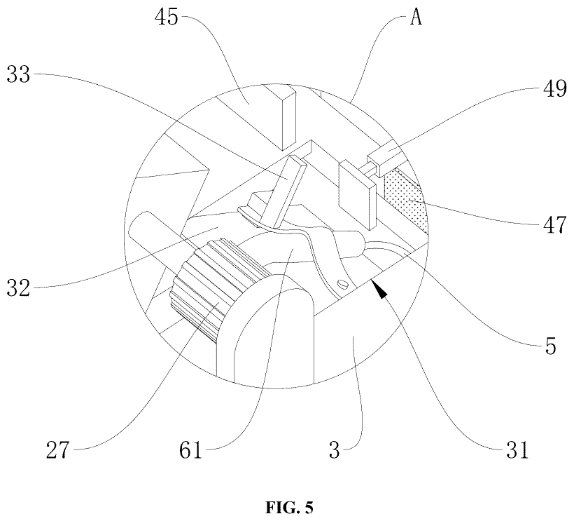

Referring to FIG. 5, when the rotating plate 32 is rotated downward, it will also drive the tail restraining assembly 33 to rotate downward, and at this time, the pull plate 332 will rotate and move to the gap between the press plate 471 and the guard plate 45, and the pull plate 332 will not bump with the press plate 471 or the guard plate 45, ensuring that the tail restraining assembly 33 can work normally.

Referring to FIGS. 19 and 23, the tail cutting assembly 4 further includes a tail releasing assembly 49. The tail releasing assembly 49 includes a second electric telescopic rod 491 and a push plate 492. The second electric telescopic rod 491 is fixedly disposed on the top of the first end of the second connecting plate 43, and the push plate 492 is disposed in front of the press plate 471 and fixedly connected to an output end of the second electric telescopic rod 491. The tail releasing assembly 49 can push the resilient metal sheet 331 out of the clamping slot 3331 to release the tail 61. Referring to FIGS. 5 and 9, a top surface of the pull plate 332 is higher than a top surface of the press plate 471, a bottom surface of the push plate 492 is lower than the top surface of the press plate 471, and a width of the push plate 492 is greater than a width of the pull plate 332.

When the protruding plate 411 drives the press plate 471 to move and the gauze 472 presses against the cut of the tail 61, the second connecting plate 43 drives the tail releasing assembly 49 to move just to a rear side of the pull plate 332. After the compression hemostasis to the tail 61 is finished, the tail releasing assembly 49 works, where the second electric telescopic rod 491 extends first to drive the push plate 492 to move forward, and the push plate 492 comes into contact with the pull plate 332, and pushes the pull plate 332 to move forward. The pull plate 332 drives the resilient metal sheet 331 to rotate while moving forward, the resilient metal sheet 331 leaves the clamping slot 3331 while rotating, and thus the tail 61 is released, and then the mouse 6 can move and leave the mouse holding tube 2. Referring to FIG. 24, the bottom surface of the push plate 492 is higher than the bottom of the cutting blade 44, so that the push plate 492 does not come into contact with the tail 61 when it moves forward.

Referring to FIGS. 1 and 2 and FIGS. 11 and 12, the carrier frame 1 includes a confining plate 11 and a support plate 12. The confining plate 11 has an opening front end surface, a second notch 111 is formed at the top of a rear end of the confining plate 11, and the support plate 12 is disposed below the rear end of the mouse holding tube 2. A positioning frame is disposed at a rear end of the carrier frame 1 and located below the support plate 12, and the first electric telescopic rod 42 is arranged horizontally and is fixedly mounted on the positioning frame. Referring to FIGS. 1 to 3 and FIG. 13, the mouse holding tube 2 is provided with a curved cover plate 22, and the curved cover plate 22 is hinged to the mouse holding tube 2. A third notch 23 is formed in the bottom surface of the front end of the mouse holding tube 2, a second carrier plate 24 is disposed in the third notch 23, and the carrier frame 1 is provided with a second drive assembly 13 configured to drive the second carrier plate 24 to move up and down.

Referring to FIGS. 2 and 3, the process of placing the mouse 6 into the mouse holding tube 2 includes firstly pushing the curved cover plate 22 to rotate to the left side of the mouse holding tube 2, placing the body of the mouse 6 on the second carrier plate 24, and then reversely rotating the curved cover plate 22 to the top of the mouse holding tube 2 to restrain the body of the mouse 6 by the curved cover plate 22 from above. In this embodiment, the curved cover plate 22 and the mouse holding tube 2 are respectively provided with a male buckle and a female buckle (not shown in the figures) that match with each other. When the curved cover plate 22 and the mouse holding tube 2 are combined, the male buckle and the female buckle are engaged together, the curved cover plate 22 cannot be rotated, the mouse 6 cannot move freely, and accordingly, the mouse 6 will not leave the mouse holding tube 2 in the process of performing the tail clipping for sampling blood of the mouse 6.

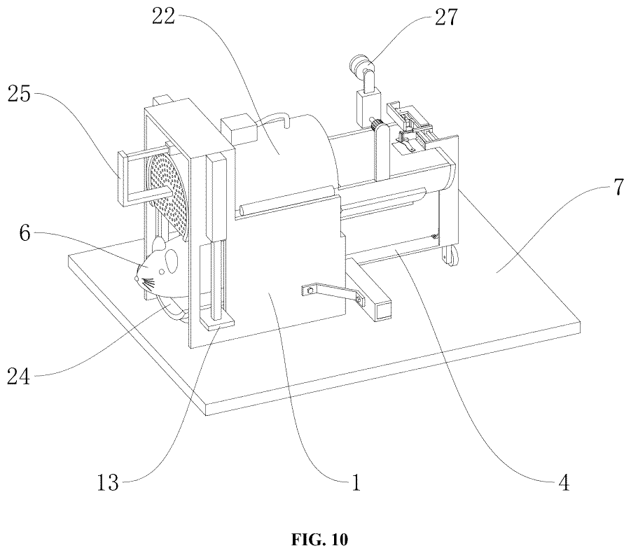

Referring to FIGS. 9 and 10, when the tail releasing assembly 49 works and the tail 61 is released, the second drive assembly 13 works to drive the second carrier plate 24 to move downward. The mouse 6 follows the second carrier plate 24 and moves downward together, and the mouse 6 moves through the third notch 23 to be below the mouse holding tube 2. Since only a front end surface of the confining plate 11 is open, the mouse 6 can only leave the carrier frame 1 from the front side of the confining plate 11 under the restraining of the confining plate 11, thus bringing convenience for the blood collector to grasp the mouse 6. After the mouse 6 leaves the carrier frame 1, the first electric telescopic rod 42 retracts to drive the first connecting plate 41 to move to the left side until the tail cutting assembly 4 returns to the initial position. At this point, the blocking plate 48 moves to the left side to expose the opening at the rear end of the mouse holding tube 2, and the centrifuge tube 5, which contains the collected the blood of the mouse 6, can be removed from the clamping hoop 29.

Referring to FIG. 1 and FIGS. 10 to 12, both the left and right sidewalls of the front end of the confining plate 11 are provided with through slots 112, and the second drive assembly 13 includes two third electric telescopic poles 131 and a drive plate 132. The two third electric telescopic rods 131 are fixedly disposed above the two through slots 112 respectively, and are fixedly connected to the confining plate 11. Output ends of the third electric telescopic rods 131 face downward. The drive plate 132 is disposed inside the front end of the confining plate 11 and is located below the second carrier plate 24. The middle of the drive plate 132 is fixedly connected to the second carrier plate 24. Two ends of the drive plate 132 extend out of the confining plate 11 from the two through slots 112 respectively. The output ends of the two third electric telescopic rods 131 are fixedly connected to the two ends of the drive plate 132, respectively.

Referring to FIGS. 10 to 12, when the tail 61 of the mouse is released, the two third electric telescopic rods 131 extend to drive the second carrier plate 24 to move downward through the drive plate 132, and then the second carrier plate 24 moves downward together with the mouse 6. After the mouse leaves the carrier frame 1, the two third electric telescopic rods 131 retract to drive the second carrier plate 24 by the driving plate 132 to move upward to the original position.

Referring to FIG. 2, FIGS. 4 to 6, and FIG. 10, the present disclosure further includes a body adapting assembly 25, a body retraining assembly 26 and a tail squeezing assembly 27. The body adapting assembly 25 is located at the front end of the mouse holding tube 2, and can adjust the size of the internal space of the mouse holding tube 2 to adapt to mice 6 having different body sizes. Since the body retraining assembly 26 is disposed on the curved cover plate 22, the body of the mouse 6 is fixed under the curved cover plate 22 and cannot move, preventing the mouse 6 from moving during the tail clipping for sampling blood. The tail squeezing assembly 27 is disposed on the top of the mouse holding tube 2 and located on a rear side of the curved cover plate 22. The tail squeezing assembly 27 can press on the tail 61 such that the blood flows out quickly from the cut of the tail 61.

Referring to FIGS. 1, 6, 10, and 14, the body adapting assembly 25 includes a mouse blocking plate 251, a fourth electric telescoping rod 252, and a third connecting plate 253. The mouse blocking plate 251 is disposed inside the front end of the mouse holding tube. The mouse blocking plate 251 is capable of sliding along an inner wall of the mouse holding tube as well as a top surface of the second carrier plate 24. A fourth electric telescopic rod 252 is fixedly disposed at the top of the front end of the mouse holding tube 2. An output end of the fourth electric telescopic rod 252 faces forward. The third connecting plate 253 is L-shaped. A first end of the third connecting plate 253 is fixedly connected to the output end of the fourth electric telescopic rod 252, and a second end of the third connecting plate 253 is fixedly connected to the mouse blocking plate 251.

Referring to FIG. 1, before the mouse 6 is placed into the mouse holding tube 2, the fourth electric telescopic rod 252 extends or retracts to drive the mouse blocking plate 251 to move back and forth to change the position of the mouse blocking plate 251, so as to change the size of the space of a rear side of the mouse blocking plate 251 in the mouse holding tube 2 to adapt to mice 6 having different body sizes. Referring to FIG. 6, after the mouse 6 is placed into the mouse holding tube 2, the mouse blocking plate 251 is just in front of the tip of the nose of the mouse 6, at which time the mouse 6 cannot move forward under the restraint of the mouse blocking plate 251. The mouse blocking plate 251 is provided with multiple air holes 2511 evenly distributed. With the air holes 2511, the air inside and outside of the mouse holding tube 2 may be circulated smoothly to ensure that the mouse 6 can breathe normally. Referring to FIG. 10, when the tail 61 is released, the fourth electric telescopic rod 252 extends to drive the mouse blocking plate 251 to move forward, so that the space of the front of the mouse 6 becomes larger, and the mouse is guided to move forward, at which time the tail 61 of the mouse 6 naturally leaves from the underside of the resilient metal sheet 331. Then, the second drive assembly 13 works to drive the second carrier plate 24 and the mouse 6 to move downward, and ultimately causing the mouse 6 to leave the mouse holding tube 2 and the carrier frame 1.

Referring to FIGS. 2 and 3 and FIG. 6, the body restraining assembly 26 includes an air pump 261, an air bag 262, and an air pipe 263. The air pump 261 is fixedly disposed on the top of the curved cover 22, and the air bag 262 is fixedly disposed on an inner wall of the curved cover 22. A first end of the air pipe 263 is connected to an air outlet end of the air pump 261, and a second end of the air pipe 263 is connected to the air bag 262. The body of the mouse 6 is placed on the second carrier plate 24, and the body restraining assembly 26 works after the male buckle and the female buckle are engaged together. The air pump 261 pumps air into the air bag 262 through the air pipe 263, the air bag 262 expands, and after a gap between the body of the mouse 6 and the curved cover plate 22 is filled with the air bag 262, the air pump 261 stops pumping air, and the mouse 6 is restricted from moving by the air bag 262. The air bag 262 is provided with an electric control valve (not shown in the figures). When the tail 61 is released, the electric control valve is started, air in the air bag 262 is then released, and the air bag 262 deflates and shrinks, no longer restraining the body of the mouse 6. When the mouse 6 leaves the mouse holding tube 2 and the carrier frame 1, the male buckle and the female buckle are separated, pushing the curved cover plate 22 to rotate to the left side of the mouse holding tube 2.

Referring to FIGS. 1 and 2 and FIG. 15, the tail squeezing assembly 27 includes a vertical plate 271, a second motor 272, a rotating box 273, a sliding plate 274, a spring 275, a press rod 276, and a pinch roller 277. The bottom of the vertical plate 271 is fixedly connected to the mouse holding tube 2, and the second motor 272 is fixedly disposed on the top of the vertical plate 271; The rotating box 273 is fixedly disposed at an output end of the second motor 272. The sliding plate 274 is disposed within the rotating box 273 and is capable of sliding along an inner wall of the rotating box 273. The spring 275 is disposed within the rotating box 273 and is located on one side of the sliding plate 274. Two ends of the spring 275 are fixedly connected to the inner wall of the sliding plate 274 and the inner wall of the rotating box 273, respectively. The press rod 276 is disposed on the other side of the sliding plate 274, and the rotating box 273 is provided with a through hole 2731. A first end of the press rod 276 is fixedly connected to the sliding plate 274, and a second end of the press rod 276 penetrates the through hole to extend out of the rotating box 273. The pinch roller 277 is disposed on the second end of the press rod 276 and is rotationally connected to the press rod 276. The pinch roller 277 is provided with a curved groove 2771.

Referring to FIGS. 3 and 4, when both the body and the tail 61 of the mouse 6 are fixedly restrained, the pinch roller 277 is located at the top of the tail squeezing assembly 27, the pinch roller 277 is disposed on the side away from the tail 61 and is not in contact with the tail 61, and here the tail squeezing assembly 27 does not work. Referring to FIGS. 4 to 6, when the mouse 6 is subjected to the tail clipping for sampling blood, the tail squeezing assembly 27 starts working. The second motor 272 drives the rotating box 273 to rotate, the rotating box 273 drives the press rod 276 to rotate through the sliding plate 274, and then the press rod 276 drives the pinch roller 277 to rotate. When the pinch roller 277 moves to the bottom of the tail squeezing assembly 27, the tail 61 enters the curved groove 2771, and the pinch roller 277 exerts pressure on the tail 61, so that the blood flows out of the cut of the tail 61 quickly. Referring to FIGS. 8 and 10, when the blood of the mouse 6 has been collected by means of the centrifuge tube 5, the pinch roller 277 re-moves to the top of the tail squeezing assembly 27, and the tail squeezing assembly 27 stops working.

Since the pinch roller 277 moves in circle, the distance between the pinch roller 277 and the rotating box 273 needs to be variable in order to ensure that the pinch roller 277 does not cause harm to the tail 61 during its movement.

In this embodiment, when the pinch roller 277 just starts to come into contact with the tail 61, the tail 61 pushes the pinch roller 277 to be close to the rotating box 273, and then the pinch roller 277 drives the press rod 276 to slide along an inner wall of the through hole 2731. The press rod 276 drives the sliding plate 274 to slide along an inner wall of the rotating box 273, the sliding plate 274 exerts pressure on the spring 275, and the spring 275 retracts to store resilient potential energy. When the pinch roller 277 gradually leaves the tail 61, the spring 275 extends to release the resilient potential energy to push the sliding plate 274 to slide outward, and ultimately to drive the pinch roller 277 to be away from the rotating box 273 and return to its original position. Therefore, when the pinch roller 277 comes into next contact with the tail 61, the pinch roller 277 can exert sufficient pressure to the tail 61.

It is apparent to those skilled in the art that the present disclosure is not limited to the details of the above exemplary embodiments, and that the present disclosure can be implemented in other specific forms without departing from the spirit or basic features of the present disclosure. Accordingly, the embodiments should be regarded in all points of view as exemplary and not restrictive, the scope of the present disclosure being defined by the appended claims rather than the foregoing description, and it is therefore intended that all changes falling within the meaning and scope of equivalent elements of the claims should be included in the present disclosure. Any reference sign in the claims should not be considered as limiting the involved claims.

In addition, it should be understood that although this specification is described in accordance with the embodiments, not each embodiment contains only one independent technical solution, this description of the specification is only for the sake of clarity, those skilled in the art should take the specification as a whole, and the technical solutions in the various embodiments may be combined appropriately to form other embodiments that can be understood by those skilled in the art.

What is claimed is:

1. A full-automatic mouse blood sampling apparatus, comprising:
a carrier frame;
a mouse holding tube disposed on a top of the carrier frame and having a first notch formed at a top of a rear end of the mouse holding tube;
a first carrier plate disposed inside the rear end of the mouse holding tube, wherein a blood sampling port is provided in a top surface of the first carrier plate, a rotating plate is disposed inside the blood sampling port, a front end of the rotating plate is rotationally connected to a side wall of the blood sampling port, a tail restraining assembly is disposed on a top surface of the rotating plate, and a first drive assembly configured to drive the rotating plate to rotate is disposed below the first carrier plate;
a tail cutting assembly disposed on a rear side of the mouse holding tube; and
a centrifuge tube disposed below a rear end of the rotating plate.

2. The full-automatic mouse blood sampling apparatus of claim 1, wherein the tail cutting assembly comprises:
a first connecting plate with a first end located below the mouse holding tube and a second end located above the mouse holding tube;
a first electric telescopic rod fixedly disposed on the carrier frame, an output end of the first electric telescopic rod being fixedly connected to the first end of the first connecting plate;
a second connecting plate disposed on the second end of the first connecting plate and provided with a cutting blade and a guard plate;
a severed-tail cleaning assembly disposed on the second end of the first connecting plate, a collection box being disposed on the first carrier plate and having an opening top surface;
a compression hemostasis assembly disposed on the second end of the first connecting plate; and
a blocking plate disposed on the second end of the first connecting plate.

3. The full-automatic mouse blood sampling apparatus of claim 2, wherein the second end of the first connecting plate is provided with a protruding plate, a first end of the protruding plate is fixedly connected to the first connecting plate, and a second end of the protruding plate extends above the first carrier plate;
the severed-tail cleaning assembly comprises a push rod and a stop lever, a first end of the push rod is fixedly connected to the protruding plate, a second end of the push rod is fixedly provided with a push block, the push block has a T-shaped cross-section, the stop lever is disposed on a rear side of the push rod, a gap is formed between the stop lever and the push rod, the stop lever has a length greater than that of the push rod, a first end of the stop lever is fixedly connected to the protruding plate, and a second end of the stop lever is located on a left side of the push block; and
the collection box is located in front of the stop lever and is removably connected to the first connecting plate.

4. The full-automatic mouse blood sampling apparatus of claim 3, wherein the second connecting plate is located above the severed-tail cleaning assembly, a first end of the second connecting plate is fixedly connected to a middle of the push rod and stop lever, the cutting blade and the guard plate are located on a second end of the second connecting plate, the guard plate is located between the cutting blade and the tail restraining assembly, the guard plate has an L-shaped cross-section, and the guard plate and the cutting blade are removably connected to the second connecting plate.

5. The full-automatic mouse blood sampling apparatus of claim 3, wherein the blocking plate is disposed below the protruding plate and is fixedly connected to a bottom of the protruding plate, a shape of the blocking plate matches with a shape of a cross-section of the rear end of the mouse holding tube, a length of the blocking plate is greater than a width of the rear end of the mouse holding tube, and a front surface of the blocking plate is capable of sliding along a rear end surface of the mouse holding tube.

6. The full-automatic mouse blood sampling apparatus of claim 3, wherein the compression hemostasis assembly comprises a press plate disposed in front of the push rod, a gap is formed between the press plate and the push rod, the press plate is fixedly connected to the protruding plate, gauze is disposed on a front side wall of the press plate, and a gap is formed between the press plate and the guard plate.

7. The full-automatic mouse blood sampling apparatus of claim 6, wherein the tail restraining assembly comprises:
a resilient metal sheet disposed on one side of the top surface of the rotating plate, a first end of the resilient metal sheet being rotationally connected to the rotating plate, a pull plate being disposed on a top surface of the resilient metal sheet, and a top surface of the pull plate being higher than a top surface of the press plate; and
a limiting block fixedly disposed on an other side of the top surface of the rotating plate, a front side wall of the limiting block being provided with a clamping slot, a width of the clamping slot being gradually decreased from front to back, a width of a front end of the clamping slot being greater than a thickness of the resilient metal sheet; and
the tail cutting assembly further comprises a tail releasing assembly comprising:
a second electric telescoping rod fixedly disposed on a top of the first end of the second connecting plate; and
a push plate disposed in front of the press plate and fixedly connected to an output end of the second electric telescopic rod, a bottom surface of the push plate being lower than the top surface of the press plate, and a width of the push plate being greater than a width of the pull plate.

8. The full-automatic mouse blood sampling apparatus of claim 1, wherein the carrier frame comprises a confining plate and a support plate, the confining plate has an opening front end surface, a second notch is formed at a top of a rear end of the confining plate, a front end of the mouse holding tube is located within the confining plate and fixedly connected to the confining plate, and the rear end of the mouse holding tube extends out of the confining plate through the second notch; and
the support plate is disposed below the rear end of the mouse holding tube, a top of the support plate is fixedly connected to the mouse holding tube, and a bottom of the support plate is fixedly connected to the confining plate.

9. The full-automatic mouse blood sampling apparatus of claim 8, wherein the mouse holding tube is provided with a curved cover plate, the curved cover plate is disposed in the first notch and located in a middle of the mouse holding tube, and the curved cover plate is hinged to the mouse holding tube; and
a third notch is formed in a bottom surface of the front end of the mouse holding tube, a second carrier plate is disposed within the third notch, and the carrier frame is provided with a second drive assembly configured to drive the second carrier plate to move up and down.

10. The full-automatic mouse blood sampling apparatus of claim 9, wherein the full-automatic mouse blood sampling apparatus further comprises:
a body adapting assembly disposed at the front end of the mouse holding tube;
a body restraining assembly disposed on the curved cover plate; and
a tail squeezing assembly disposed on a top of the mouse holding tube and located on a rear side of the curved cover plate.
